(19) United States
(12) Patent Application Publication
(10) Pub. No.: US 2014/0356805 A1
(43) Pub. Date: Dec. 4, 2014

(54) RADIOGRAPHIC GUIDE WITH RADIO-OPAQUE FEATURES

(71) Applicants: Charles Louis Marie Deville, Asnieres sur-Seine (FR); Sylvie Marie Gisele Bothorel, Paris (FR); Philippe Congy, Meaux (FR)

(72) Inventors: Charles Louis Marie Deville, Asnieres sur-Seine (FR); Sylvie Marie Gisele Bothorel, Paris (FR); Philippe Congy, Meaux (FR)

(73) Assignee: TROPHY, Marne La Vallee Cedex 2 (FR)

(21) Appl. No.: 14/372,035

(22) PCT Filed: Jan. 18, 2012
(86) PCT No.: PCT/IB2012/000214

§ 371 Date: Jul. 14, 2014

(51) Int. Cl. A61C 1/08; A61C 19/04; A61B 19/00; A61B 6/14; A61B 6/12

(52) U.S. Cl. CPC A61C 1/084; A61B 6/145; A61B 6/12; A61B 19/54; A61C 19/04; A61B 2019/5466
USPC 433/29; 433/215

(57) ABSTRACT

A blank for forming a radiographic guide is formed from a material substantially translucent to x-rays and has external surfaces. The blank encases portions of one or more radio-opaque features, wherein the one or more radio-opaque features are embedded within the blank and extend at least between the external surfaces of the blank.

### FIELD OF THE INVENTION

The invention relates generally to radiographic imaging and more particularly to a guide apparatus and method for assisting in visualization and installation of a dental prosthetic device.

### BACKGROUND OF THE INVENTION

Proper installation of dental implants requires a considerable amount of care and planning. For installing an implant in the patient's jawbone, for example, the dentist first drills a hole in the patient's jawbone at the implant site using a handheld drill. During the drilling procedure, there are a number of factors to consider to reduce risk to anatomic structures such as to nerves, to existing tooth roots, or to nearby blood vessels. The jawbone structure itself needs sufficient width for implant support. Proper implant definition depends on the definition of the dental restoration that will be positioned on the implant and, consequently, is also dependent on the shape and position of the neighboring teeth in the patient's mouth. The orientation, depth, and diameter of the hole need to be defined carefully, depending on each of these considerations.

To assist in installation planning and execution for dental implantology, a volume image of the mouth anatomy, such as an image from a cone beam computed tomography (CBCT) scan can be employed to provide detailed information about the patient's anatomy. This scan produces accurate three dimensional views of the patient's actual anatomy and reveals features and structures that can be of particular interest for the drilling procedure. To assist the dentist in implant planning, a prosthesis replica, called a custom radiographic guide, can be fabricated for a fully or partially edentulous patient. Because of the difficulty of obtaining the needed information from a single volume image, a “double-scan” protocol has been adapted to help in preparing for this installation task. In this process, the radiographic guide is inserted into the patient's mouth and one CT scan is obtained. Another CT scan is obtained of the radiographic guide itself, removed from the patient. Data from the two CT scans can then be correlated and used for computer-based planning of the installation surgery and related techniques, possibly including preparation of a surgical template, as described in the article by van Steenberghe, Glauser, Blomback, Andersson, Schutyser, Petersson, and Wendelhag, entitled “A Computed Tomographic Scan-Derived Customized Surgical Template and Fixed Prosthesis for Flapless Surgery and Immediate Loading of Implants in Fully Edentulous Maxillae: A Prospective Multicenter Study”, Clinical Implant Dentistry and Related Research, Volume 7, Supplement 1 (2005), pp. S111-S119.

In order to make use of the CT scan information, the two scans that are obtained are matched to each other, so that the corresponding information can be digitally superimposed. Reference markers are positioned on the radiographic guide so that the two images can be properly correlated. In conventional practice, radio-opaque gutta percha markers are used for this purpose. The gutta percha markers on the radiographic guide are then used as reference points to perform a match of the first CT scan and the second CT scan. Data from the first and second CT scans can then be matched and used for computer based planning of a subsequent surgery, including production of the surgical template that provides suitable bores that guide the drilling of holes in the patient's jawbone prior to mounting the dental implants to which dental restorations are fixed.

While conventional methods may have achieved a level of success for providing ways to match the different CT scans, there is acknowledged to be some need for improvement. The gutta percha markers are manually attached to outer surfaces of the radiographic guide, in a process that can be tedious and depends upon technician expertise to select the three or more points best suited for positional reference. Typically, holes are drilled into the guide and the radio-opaque material, in the form of small spheres or rods, inserted into the holes. In some cases, surface re-finishing is then needed in order to remove protruding material. Further, there can be little control over the size of the radio-opaque markers, complicating the task of proper identification and alignment.

Thus, it can be seen that there is a need for improved marking methods that provide radio-opaque features for a radiographic guide.

### SUMMARY OF THE INVENTION

Embodiments of the present invention address the problem of providing radio-opaque markers for a radiographic guide, such as a radiographic guide used for implant planning or appliances and similar devices. With this goal in mind, embodiments of the present invention provide a material having embedded radio-opaque features, wherein the material can be machined or otherwise shaped to form the radiographic guide with clearly identifiable markers. Advantageously, separate steps for application of markers and related post-finishing operations are no longer needed when using the method of the present invention.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to one aspect of the invention, there is provided a blank for forming a radiographic guide for dental implantology, the blank formed from a material substantially translucent to x-rays and having external surfaces, the blank further encasing portions of one or more radio-opaque features, wherein the one or more radio-opaque features extend at least between the external surfaces of the blank.

From an alternate aspect, the present invention provides a method for combining image information from two or more volume images of a radiographic guide, the method comprising: obtaining the first volume image with the guide in the patient's mouth; obtaining the second volume image of the guide only, removed from the patient; registering the first volume image with the second volume image according to radio-opaque features that are encased by a translucent medium of the radiographic guide and that extend within the radiographic guide, between external surfaces; and displaying the registered images.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other.

FIG. 1 shows a conventional radiographic guide with a number of markers.

FIG. 2A shows a perspective view of a blank formed with one or more embedded radio-opaque features.

FIG. 2B shows a perspective partially transparent view of a blank with one or more embedded radio-opaque features.

FIG. 2C shows a perspective partially transparent view of a blank with one or more embedded radio-opaque features in a radial arrangement.

FIG. 3A shows a perspective view of a machined blank formed from the blank of FIG. 2A.

FIG. 3B shows a perspective partially transparent view of the machined blank of FIG. 3A.

FIG. 4 is a logic flow diagram showing the process for developing an implantology plan according to an embodiment of the present invention.

FIG. 5 is a schematic diagram that shows a dental imaging system for processing image data obtained using the radiographic guide.

### DETAILED DESCRIPTION OF THE INVENTION

The following is a detailed description of the preferred embodiments of the invention, reference being made to the drawings in which the same reference numerals identify the same elements of structure in each of the several figures.

The term “radio-opaque” refers to materials having sufficient density to block radiation in radiographic imaging, including metals and other high-density materials for example. The term “translucent” refers to materials of less density that exhibit some transparency, but present some amount of blockage or scattering to imaging radiation, so that their presence can be readily perceived in the image, without blocking the visibility of denser materials. A translucent element that encases a radio-opaque object allows the encased radio-opaque object to be readily perceived in a radiographic image, but without loss of at least the outline definition of the encasing translucent element. The degree of perceptibility of a translucent element is determined by a number of factors including the material used and the variable amount of radiant energy applied; hence, the term “substantially translucent” is used.

The term “blank” has its standard meaning as an intermediate element in a fabrication process, a piece of material that is ready to be made into a finished product. A blank typically has standard size, shape, and surface characteristics and is further processed or customized to specific requirements, such as to fit in a position of the patient's mouth, in a finishing process.

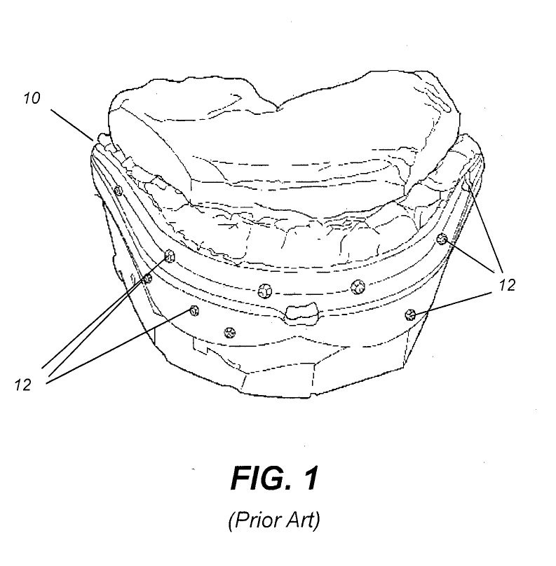

The perspective view of FIG. 1 shows a conventional radiographic guide 10 used for dental implantology. Guide 10 is prepared for CT imaging and having a number of radio-opaque markers 12 applied to its surface using conventional methods. Markers 12 are features typically formed from gutta percha or other suitable material, such as barium sulfate or kavite, for example. As noted in the background section, markers 12 often require a significant amount of work for insertion or application and may require additional finishing steps, depending on their location. Embodiments of the present invention address this problem by providing a base material that is translucent to x-rays and that has embedded marker features, eliminating technician steps for application and finishing and simplifying the task of matching two or more CT images.

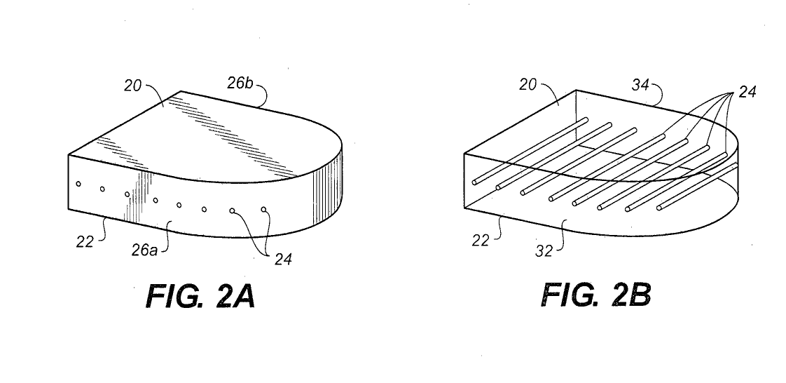

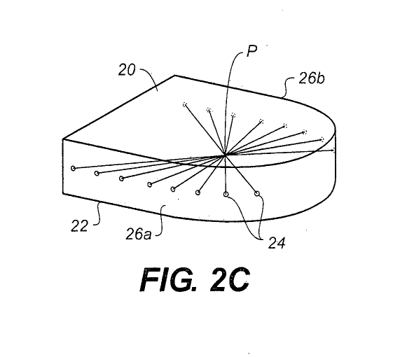

The perspective view of FIG. 2A and transparent view of FIG. 2B show a blank 20, an intermediate body that is initially formed in an approximate shape and is ready to be shaped to final form in final fabrication steps, formed from a medium 22 that is translucent to x-ray radiation and with one or more embedded radio-opaque features 24. In the embodiment shown, radio-opaque features 24 are circularly cylindrical elements that are formed within blank 20 and extend between external surfaces, shown as opposed surfaces 26a and 26b in FIG. 2A. According to an embodiment of the present invention, translucent medium 22 is PMMA (poly(methyl methacrylate)), a translucent thermoplastic having glass-like optical properties. Other plastics can be used. Still other materials are suitable, including composite materials and materials that are even less transparent to visible light. Blank 20 can be molded as a single piece or shaped from a larger block, for example.

It is noted that radio-opaque features 24 embedded within blank 20 can be formed in any shape and orientation that is suitable for detection in an x-ray. Features 24 in the exemplary FIG. 2A and 2B embodiment are circularly cylindrical, of the same diameter, and disposed in parallel, evenly spaced apart from each other and extending fully from one surface 32 to the other surface 34. Each of features 24 is in the same plane. While there are advantages to the parallel arrangement shown, any of a number of other arrangements are possible and may be suitable for different types of prosthetic devices. For example, uneven spacing may be useful, as well as orientation in an alternate direction or directions, with at least some of the features 24 of different diameter or not in parallel. As is shown by way of example in FIG. 2C, features can alternately be arranged in a pattern that radiates outward toward multiple surfaces from a central point P. Features can also be randomly arranged. Conventional practice uses spherical markers, but other shapes could be used. An overmolding method can be used for forming the blank.

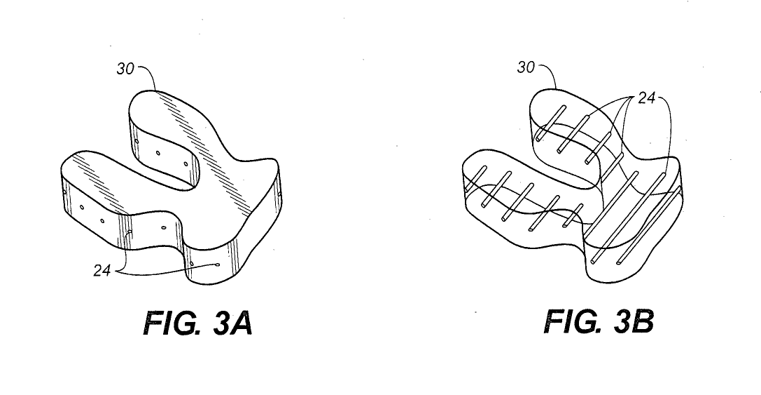

Blank 20 of FIG. 2A, prepared in this manner, is then machined in a finishing process to form a prosthetic device with embedded radio-opaque markers that extend through the device from one external surface to another, suitable for a particular patient. The perspective views of FIGS. 3A and 3B show overall and partially transparent views of a machined blank 30 that will be further shaped to form radiographic guides 10 for dental implantology, as was shown in FIG. 1, with the addition of embedded marker features 24. It can be appreciated that no separate drilling and depositing operations are required to provide radio-opaque markers extending between opposed external surfaces with this arrangement. Notably, radio-opaque materials, because they are embedded within blank 30, extend through the finished radiographic guide, rather than only being applied to the surface. Volume images can be more readily matched using marker features 24 having this arrangement, extending from one surface to another within the volume of the guide.

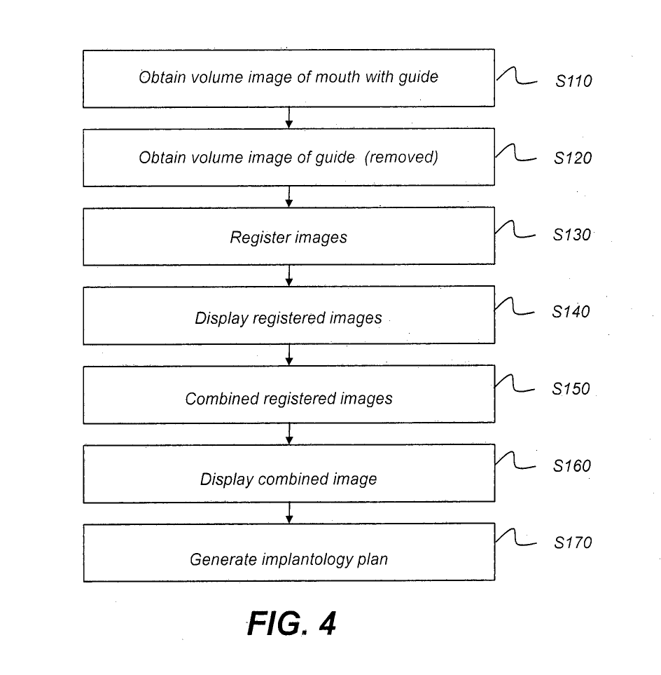

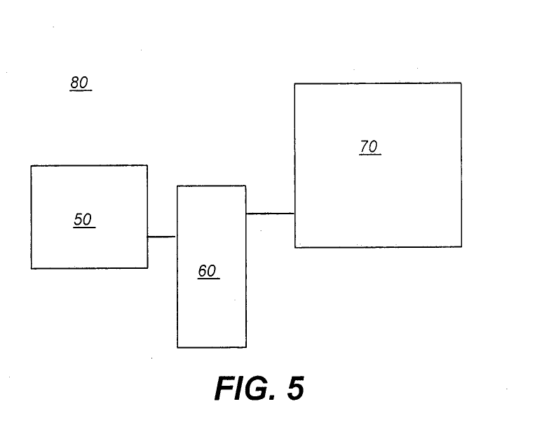

The logic flow diagram of FIG. 4 shows steps used for combining image information from two or more volume images using a radiographic guide and generating an implantology plan for dental implant procedure. FIG. 5 is a schematic block diagram that shows a dental imaging system 80 for this process. A dental imaging apparatus 70 for volume imaging, such as a CBCT imaging apparatus, is in signal communication with a computer 60 that executes image acquisition and processing logic. A display 50 shows processing results. In a first imaging step S110, a first volume image is obtained with the guide in the patient's mouth. In a second imaging step S120, the guide is removed from the patient's mouth and a second volume image is obtained, of the guide only. The first and second images are then co-registered in a registration step S130, using the radio-opaque features that are embedded within the guide. An optional display step S140 displays the registered images. A subsequent combination step S150 combines the first and second images to form a combined image, using techniques well known to those skilled in the image processing arts. An optional display step S160 then displays the combined image results.

A plan generation step S170 uses the imaging results for developing an implantology plan. This determines, for example, where holes are to be drilled and various parameters such as hole width, depth, and other characteristics. Plan generation step S170 can be performed using programmed computer logic or may be manually performed by the practitioner, using the images obtained from this process.

Consistent with an embodiment of the present invention, computer 60 or other type of logic processing device executes a program with stored instructions that perform on image data accessed from an electronic memory. As can be appreciated by those skilled in the image processing arts, a computer program of an embodiment of the present invention can be utilized by a suitable, general-purpose computer system, such as a personal computer or workstation, as well as by a microprocessor or other dedicated processor or programmable logic device. However, many other types of computer systems can be used to execute the computer program of the present invention, including networked processors. The computer program for performing the method of the present invention may be stored in a computer readable storage medium. This medium may comprise, for example; magnetic storage media such as a magnetic disk (such as a hard drive) or magnetic tape or other portable type of magnetic disk; optical storage media such as an optical disc, optical tape, or machine readable bar code; solid state electronic storage devices such as random access memory (RAM), or read only memory (ROM); or any other physical device or medium employed to store a computer program. The computer program for performing the method of the present invention may also be stored on computer readable storage medium that is connected to the image processor by way of the internet or other communication medium. Those skilled in the art will readily recognize that the equivalent of such a computer program product may also be constructed in hardware.

It is understood that the computer program product of the present invention may make use of various image manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present invention, are not specifically shown or described herein and may be selected from such algorithms, systems, hardware, components and elements known in the art.

It is noted that the term “memory”, equivalent to “computer-accessible memory” in the context of the present disclosure, can refer to any type of temporary or more enduring data storage workspace used for storing and operating upon image data and accessible to a computer system. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing processes and for recording entered values, such as seed points, or storing intermediate and final results of calculations and other processing. Computer-accessible memory can be volatile, non-volatile, or a hybrid combination of volatile and non-volatile types. Computer-accessible memory of various types is provided on different components throughout the system for storing or recording, processing, transferring, and displaying data, and for other functions.

The subject matter of the present invention relates to digital image acquisition and processing for volume images, which is understood to mean technologies that digitally process one or more digital images to recognize and thereby assign useful meaning to human understandable objects, attributes or conditions, and then to utilize the results obtained in the further processing of the digital image content.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A blank for forming a radiographic guide, the blank formed from a material that is substantially translucent to x-rays and having external surfaces, the blank encasing portions of one or more radio-opaque features, wherein the one or more radio-opaque features are embedded within the blank and extend at least between the external surfaces of the blank.

2. The blank of claim 1 wherein the one or more radio-opaque features is formed from one or more of the following: gutta percha, barium sulfate, and kavite.

3. The blank of claim 1 wherein at least two of the one or more radio-opaque features are parallel.

4. The blank of claim 1 wherein the blank comprises a polymethyl(methacrylate).

5. The blank of claim 1 wherein the one or more radio-opaque features are in the same plane.

6. The blank of claim 1 wherein the blank material comprises a plastic.

7. The blank of claim 1 wherein the one or more radio-opaque features are cylindrical.

8. The blank of claim 1 wherein the one or more radio-opaque features are further disposed in a pattern that radiates outward from a center portion of the blank.

9. The blank of claim 1 wherein the portions of one or more radio-opaque features are encased by over-molding.

10. A radiographic guide formed from a blank that comprises a substantially translucent material and has first and second external surfaces,
wherein the translucent material encases portions of one or more radio-opaque features, wherein the features extend at least between the first and second external surfaces of the blank.

11. The radiographic guide of claim 10 wherein the portions of one or more radio-opaque features are encased by over-molding.

12. The radiographic guide of claim 10 wherein at least one of the external surfaces of the blank is formed by milling.

13. The radiographic guide of claim 10 wherein the one or more radio-opaque features are cylindrical.

14. The radiographic guide of claim 10 wherein the one or more radio-opaque features are parallel.

15. The radiographic guide of claim 10 wherein the one or more radio-opaque features lie in the same plane.

16. The radiographic guide of claim 10 wherein the one or more radio-opaque features lie in two or more planes.

17. A method for combining image information from two or more volume images of a radiographic guide, the method executed at least in part by a computer, comprising:
obtaining the first volume image with the guide in the patient's mouth;
obtaining the second volume image of the guide only, the guide being removed from the patient's mouth;
registering the first volume image with the second volume image according to radio-opaque features that are encased by a substantially translucent medium of the radiographic guide and that extend within the radiographic guide, between external surfaces; and
displaying the registered images.

18. The method of claim 17 further comprising combining the registered images to form one registered volume image.

19. The method of claim 17 further comprising generating a treatment plan for a dental implant according to the registered images.
